(19) United States
(12) Patent Application Publication
(10) Pub. No.: US 2004/0102786 A1
(43) Pub. Date: May 27, 2004

(54) System for reconstructing the natural torque between the natural knee and the natural hip area

(75) Inventor: Hans Grundei, Lubeck (DE)

Correspondence Address: AKIN GUMP STRAUSS HAUER & FELD L.L.P., ONE COMMERCE SQUARE, 2005 MARKET STREET, SUITE 2200, PHILADELPHIA, PA, 19103-7013, US

(73) Assignee: ESKA Implants GmbH & Co.

(21) Appl. No.: 10/663,529

(22) Filed: Sep. 15, 2003

Related U.S. Application Data
(63) Continuation of application No. PCT/EP02/01771, filed on Feb. 20, 2002.

(30) Foreign Application Priority Data
Mar. 15, 2001 (DE) 101 13 069.4

(51) Int. Cl. A61B 17/58

(52) U.S. Cl. 606/88

(57) ABSTRACT

A system is provided for reconstructing the natural torque between the natural knee and the natural hip area, including a saw jig (2) with an exact parallelepipedal configuration, traversed by saw slots (16, 17, 18) for producing perpendicular ventral and dorsal and diagonal cuts. The front face of the saw jig can be placed on the horizontal cut of the femur (13) and detachably fixed thereto. The saw jig has a central opening (19) and is provided with coupling means for detachably coupling to a rotational jig (1). The system also includes an intramedullary pin (11), provided on the femur side, which can be connected in a pivoting manner to the rotational jig (1) and can be inserted in the femoral bone canal. The saw jig (2) can be threaded onto the intramedullary pin (11) and can then be coupled to the rotational jig (1), whereby the intramedullary pin (11) forms a pivoting axis for the saw jig (2), about which axis the latter can be pivoted plus or minus 10° about the horizontal. In addition, the system is provided with a drive mechanism (14), which pivots the saw jig (2) about the horizontal.

### CROSS-REFERENCE TO RELATED APPLICATIONS

This application is a continuation of International Application No. PCT/EP02/01771, filed Feb. 20, 2002, the disclosure of which is incorporated herein by reference.

### BACKGROUND OF THE INVENTION

The present invention relates to a system for the reconstruction of the natural torque between the natural knee and natural hip area, in an arrangement between an artificial knee joint and the natural hip area, after partial resection of the natural condyles of the knee joint in a horizontal cut.

Such a system is known from German Patent DE-197 16 300 C1. It has a nail jig having a precisely rectangular seating (bearing) block having on its end surface, seen from the ventral view, a limb protruding therefrom and pointing in the direction of the femur and that holds on its end a femur contact probe in the form of a pin standing perpendicularly on the limb, for a ventrally situated, point-like seating on the femur, the block also having on its end surface, seen from the dorsal view, at least one contact plate protruding therefrom and pointing toward the femur, for the respectively dorsally situated, point-like seating on both dorsal condyle rolls. The seating block is penetrated by pairs of bore holes, whose arrangement represents the respective size of the femur part, through which fixation nails can be placed, which secure the seating block in its position on the femur. The seating block can be withdrawn from the femur while leaving behind the fixation nails. In addition, the system provides a saw jig, having a basic shape identical to that of the seating block of the nail jig, and having an identical arrangement of the pairs of bore holes in the seating block. The saw jig can be placed on the fixation nails, which were put into place with the aid of the nail jig. Its end surfaces determine the remaining resection planes (ventral, dorsal, diagonal).

An improvement of this resection template is known from German Patent DE-197 53 236 C1. The object of this system is to form an exact reproduction of the natural torque that exists between the knee and the hip, under the precondition that the resection cut on the tibia is not a horizontal cut, but rather is an inclined cut, slanted at an angle between 3° and 5° from the horizontal, in the direction from lateral to medial. For this purpose, in contrast to the system mentioned above, this system has a wedge having a wedge angle that can be shifted between the lateral condyle rolls and the contact plate, in order to compensate a wedge angle in which the horizontal tibia cut deviates from the knee gap axis.

The system mentioned above has the disadvantage that, in practice, the surgeon has a tendency always to use the same wedge with the same wedge angle. However, this does not meet all the individual, patient-specific requirements of each case.

### BRIEF SUMMARY OF THE INVENTION

Against this background, the object of the present invention is to improve the mentioned system in such a way that it can be used in a wider range of situations, and above all can be adapted to the particular circumstances of each case.

Starting from the system mentioned above, this object is achieved by the system according to the present invention having the following components:

a saw jig in the form of an exact parallelepiped, preferably rectangular, block, which is penetrated by saw slits for the creation of perpendicular cuts ventrally and dorsally, as well as diagonal cuts, this jig being placeable with a front side on the horizontal cut of the femur and being detachably fixed there, and having a central bore hole, and being provided with coupling means for detachable coupling to a rotational jig, which grips over the saw jig and has at least one protruding contact plate pointing toward the femur, for dorsally situated, point-like seating on each of the two dorsal femur condyle rolls;

an intramedullary spike, connectable in pivotable fashion with the rotational jig at the femur, for use in the femur bone canal, the saw jig being threadable on the intramedullary spike and then couplable with the rotational jig, and wherein the intramedullary spike forms a pivot axis for the saw jig, about which this jig is pivotable in a range of ±10° about the horizontal;

a drive mechanism which pivots the saw jig about the horizontal; and

a limb connectable in detachable fashion with the functional unit made up of the saw jig and the rotational jig, and pointing toward the femur, having on its end a ventral femur contact probe in the form of a pin standing perpendicularly on the limb, for ventrally situated, point-like seating on the femur.

Advantageous improvements are described below and in the dependent claims.

With the system according to the present invention, it is possible, without using a nail jig from the prior art, to orient the saw jig on the horizontal cut of the femur and to fix it there, such that no change takes place between a nail jig and a saw jig. Rather, the system according to the present invention permits the continuous adjustment of the saw jig in the range of ±10° relative to the horizontal, due to the rotational jig and the subsequent fixing of the saw jig on the femur with at least two fixation nails. The rotational jig can then be decoupled from the saw jig, and the femur contact probe can be removed. In order to produce the perpendicular cuts ventrally and dorsally, as well as the diagonal cuts, according to an advantageous embodiment the saw jig is fastened to the femur using additional fastening means for temporary fixing. An additional fastening means can be two lateral tongues (flaps) through which bore holes are made, through which bone screws can be guided and screwed into the bone of the femur.

It is of essential importance in the system according to the present invention that the rotational jig, on which the saw jig is first threaded, is brought into a stable three-point seating on the femur with the rotational jig being pivotably connected with the intramedullary spike at the femur. The mentioned points of contact of the contact plate of the rotational jig on the rear condyle rolls form two points for this envisioned stable three-point seating. The third point is the point of contact of the femur contact probe of the functional unit made up of the saw jig and the rotational jig. This means that the femur contact probe can be connected in detachable fashion with the saw jig or with the rotational jig. Since in this state the saw jig is coupled with the rotational jig, both approaches are conceivable.

With its positively locking seating in the intramedullary bone canal, the intramedullary spike defines the pivot axis for the saw jig. Together with the ventral femur contact probe, it forms for the respective femur an unambiguously defined reference system, in which the natural torque is reproduced. The intramedullary spike is the precondition for the further components, namely the drive mechanism with whose aid the saw jig can be pivoted about the pivot axis defined by the intramedullary spike. This pivoting is the generalization of the principle known from DE 197 53 236 C1, in which the above-mentioned wedge having the predetermined wedge angle is used to compensate a wedge angle by which the horizontal tibia cut deviates from the knee gap axis. In comparison with this system, the system according to the present invention is extremely flexible, and permits individual adjustments in a wide range of ±10′ about the horizontal. In this way, compensations of all kinds of deformations and damage are possible. Even pivotings in a direction opposite to that described in DE 197 53 236 C1 are possible.

Preferably, the surgeon adjusts the compensation angle using the drive mechanism before the resection, and then guides the intramedullary spike into the opened femur canal. Due to the free pivotability of the spike on the rotational jig, this spike automatically finds its optimal position in the bone canal.

According to a preferred development, the intramedullary spike is coupled to the rotational jig by means of a cone-fit (tapered fit) seat. This permits, on the one hand, the already-mentioned free pivotability of the spike in relation to the rotational jig. On the other hand, the cone-fit seat permits an exchanging of the intramedullary spike with a spike having different dimensions. Thus, in principle, the intramedullary spike must be selected on an individual patient basis, and installed using the cone-fit seat. The diameter of the spike, in particular, plays an essential role here.

Preferably, the drive mechanism of the rotational jig for adjustment of the compensation angle can be adjusted continuously. An immediate consequence of this is the continuous adjustability of the compensation angle of the saw jig in the indicated range of ±10° from the horizontal.

It is preferred that the drive mechanism snap mechanically into predetermined adjustment positions, despite its continuous adjustability. This increases the surgeon's tactile perception of the angle by which the saw jig has already been pivoted. For example, with each additional half-degree increase of the pivot angle, a snapping or clicking can be produced in the drive mechanism, so that the surgeon is given a representation of the angle by which the saw jig has already been pivoted; this is important in view of the extremely narrow spatial conditions that are present during the operation.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there is shown in the drawings an embodiment which is presently preferred. It should be understood, however, that the invention is not limited to the precise arrangements and instrumentalities shown. In the drawings:

FIG. 1 is a front end view of the rotational jig with coupled-on saw jig, according to the invention;

FIG. 2 is a perspective view of the functional unit made up of the rotational jig and the saw jig of FIG. 1;

FIG. 3 is a plan view of the saw jig of FIGS. 1 and 2, but decoupled from the rotational jig; and

FIG. 4 is a longitudinal schematic view showing the seating of the rotational jig with coupled-on saw jig on a femur bone, after horizontal resection has taken place.

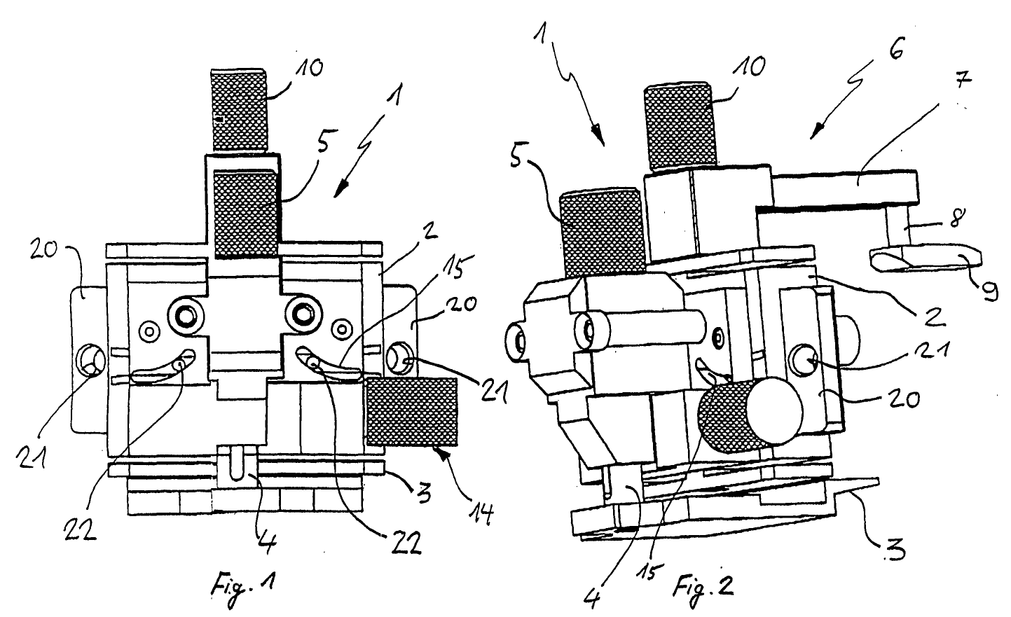

In the following, like parts are provided with the same reference numerals.

### DETAILED DESCRIPTION OF THE INVENTION

FIG. 1 shows the front view of rotational jig 1 of the system according to the present invention, with saw jig 2 coupled thereon. Rotational jig 1 has on the dorsally situated side (i.e., at bottom in FIG. 1) a contact plate 3, which comes into contact with the rear condyle rolls of the femur at only two points (FIG. 4). In the present case, contact plate 3 is connected with the rotational jig via a drive rod 4, which works together with a knurled screw 5 as a drive mechanism, so that the distance of contact plate 3 from the condyles can be varied, in such a way that the rotational jig acts in the manner of a sliding template (jig) for the determination and definition of the size of the femur part of the artificial knee joint. After determination of the size of the femur part implant, the surgeon must bring the rotational jig into a stable three-point seating with the two support points on the rear condyle rolls and contact point C. Only then is the reference system created for the torque between the knee and the hip regions of the patient. The mentioned contact points of contact plate 3 on the rear condyle rolls form two points for an intended stable three-point seating. The third point for this seating is contact point C (FIG. 4) of a femur contact probe 6 of the system. Here, femur contact probe 6 is formed as a pin 8 standing perpendicularly on a limb 7 protruding from the ventrally situated end surface of saw jig 2 and pointing toward the femur, the pin having seating shoe 9 fixed thereon.

A screw 10, with which the femur contact probe is attached to saw jig 2, makes it possible to bring femur contact probe 6 against the femur from the ventral side.

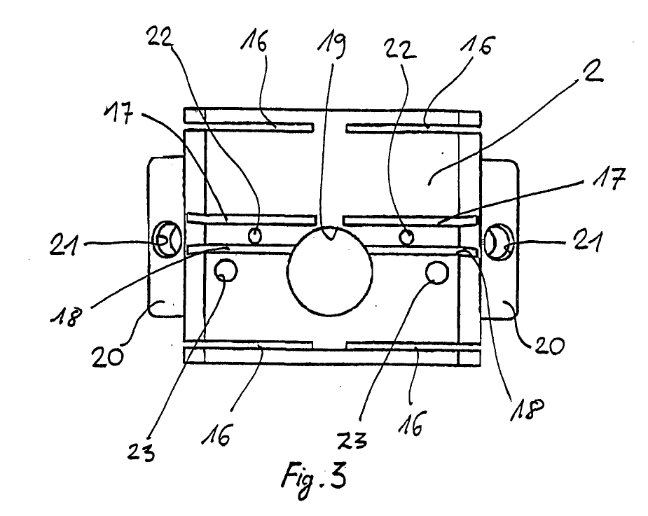

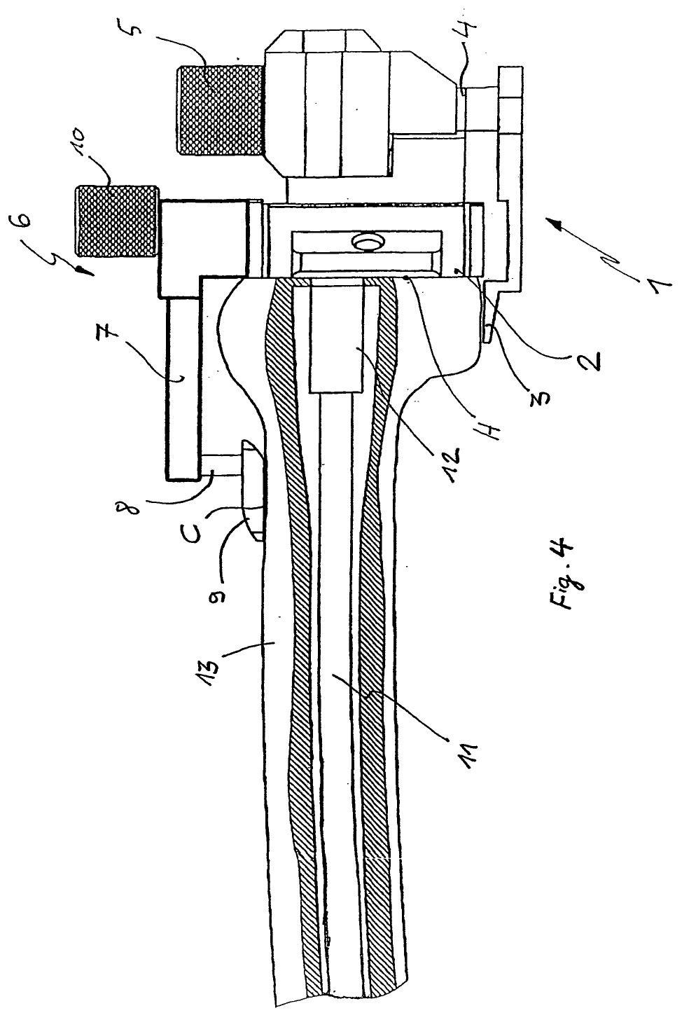

Details of saw jig 2 can be seen in FIG. 3. Saw jig 2 is configured as an exact rectangular block, and is penetrated by saw slits 16, 17 and 18. Saw slits 16 are used for the production of the perpendicular cuts ventrally and dorsally, whereas saw slits 17 and 18 are used for the production of the diagonal cuts. To the sides, i.e., laterally and medially, tongues 20 are attached, each of which is penetrated by a bore hole 21. These serve for the attachment of bone screws (not shown) for the temporary fixing of saw jig 2, adjusted correctly in its position, for the preparation of the resections, so that the position of saw jig 2 remains true in its position unchanged even during the resection cuts.

Saw jig 2 has a central opening 19 through which the intramedullary spike 111 passes, when the system according to the present invention is used.

Bore holes 22 in saw jig 2 serve for the guiding through of fixation nails (not shown), after the angular position of saw jig 2 in relation to the horizontal has been brought into the correct position by pivoting relative to the horizontal.

Openings 23 or blind holes 23 are used for temporary coupling between saw jig 2 and rotational jig 1.

As a further essential element, in FIG. 4 intramedullary spike 11 is shown. This spike is detachably connected with rotational jig 1 via a cone-fit seating, and can thus be pivoted in all directions. When the bone canal of femur 13 is opened, intramedullary spike 11 seeks the correct position in a certain sense, in that the shape of the bone canal predetermines the pivoting of spike 11 during its introduction into the bone canal. Here, the surgeon must pay attention to the correct selection of the proper spike, so that this spike comes to lie freely in the bone canal.

With this, the coordinate system for making the resection cuts is complete. The surgeon must now still determine the deviation of saw jig 2 from the horizontal, in order to carry out a compensation for any deformation or damage, but also attend to a compensation for the angle by which the horizontal tibia cut deviates from the knee gap axis. For this purpose, a pivot mechanism 14 (FIG. 1) is provided, with whose aid the surgeon can pivot saw jig 2 out of the horizontal by an angle of ±10°. This is indicated by the curvatures of longitudinal openings 15. By the actuation of drive mechanism 14, saw jig 2 is pivoted about an axis, which is determined by intramedullary spike 11.

After the desired position has been achieved, saw jig 2 is fixed on the horizontal cut of femur 13 in a known manner, using fixation nails that are hammered into the femur through bore holes 22.

The rotational jig is then decoupled from saw jig 2. Subsequently, saw jig 2 is temporarily fixed on the femur using bone screws that are placed through bore holes 21 in tongues 20, in order, as already mentioned, to make the perpendicular cuts ventrally and dorsally as well as the diagonal cuts, whereby the torque is maintained due to the use of the system according to the present invention.

In the following, the use of the system according to the present invention is briefly described:

At the beginning of the resection, horizontal seating surface H (FIG. 4) is first produced. Here, rotational jig 1 with saw jig 2 coupled thereon, which is threaded on the intramedullary spike, is placed on the horizontal seating surface H, with simultaneous introduction of intramedullary spike 11 into the bone canal of femur 13. Rotational jig 1 is here equipped with drive mechanism 4, 5, so that it can be used in the manner of a sliding template for determining the size of the femur part of the artificial knee joint. For this purpose, rotational jig 1 is brought into the mentioned stable three-point seating, so that contact plate 3 therefore touches the rear condyle rolls dorsally only at one point on each roll, and femur contact probe 6 likewise touches femur 13 at point C on the opposite side of the femur. The stable three-point seating is not adversely influenced by a direct contact of saw jig 2 with horizontal seating surface H. The pivoting of saw jig 2 in the desired direction then takes place with the aid of drive mechanism 14, 15, after which saw jig 2 is then fixed, as described, on the femur using fixation nails, after which rotational jig 1 is decoupled from saw jig 2, and likewise is removed, as is femur contact probe 6. Saw jig 2 is then fixed on the femur using bone screws, and the resection cuts are carried out.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I claim:

1. A system for reconstruction of natural torque between a natural knee joint and natural hip area, in an arrangement between an artificial knee joint and the natural hip area, after partial resection of natural condyles of the knee joint in a horizontal cut of a femur, comprising:
a saw jig (2) in a form of an exact parallelepiped block, which is penetrated by saw slits (16, 17, 18) for production of perpendicular cuts ventrally and dorsally, as well as diagonal cuts, the saw jig being placeable with a front side against the horizontal cut of the femur (13) and detachably fixed there, having a central opening (19), and being provided with a coupling for detachable coupling to a rotational jig (1), which grips over the saw jig (2) and has at least one contact plate (3) protruding toward the femur, for dorsally situated, point-like seating on each of two dorsal femur condyle rolls;
an intramedullary spike (11), pivotably connectable with the rotational jig (1) at the femur, for insertion into the femur bone canal, wherein the saw jig (2) is threadable on the intramedullary spike (11) and then couplable with the rotational jig (1), and wherein the intramedullary spike (11) forms a pivot axis for the saw jig (2), about which the saw jig is pivotable in a range of ±10° about the horizontal;
a drive mechanism (14) which pivots the saw jig (2) about the horizontal, and
a limb (7) pointing toward the femur and being detachably connectable with a functional unit comprising the saw jig (2) and the rotational jig (1), the limb having on its end a ventral femur contact probe (6) in a form of a pin (8) standing perpendicularly on the limb (7), for ventrally situated seating on the femur (13).

2. The system according to claim 1, wherein the intramedullary spike (11) is linked to the rotational jig (1) by a cone-fit seating.

3. The system according to claim 1, wherein the drive mechanism (14) of the rotational jig is continuously adjustable.

4. The system according to claim 3, wherein the drive mechanism (14) snaps into predetermined adjustment positions.

5. The system according to claim 1, wherein the saw jig (2) has additional fasteners (20) for temporary fixing on the femur.
